(19) United States
(12) Patent Application Publication
(10) Pub. No.: US 2008/0015188 A1
(43) Pub. Date: Jan. 17, 2008

(54) Stable pharmaceutical compositions of 2-aza-bicyclo(3.3.0)-octane-3-carboxylic acid derivatives

(76) Inventors: Julia Hrakovsky, Rosh Ha-Ayin (IL); Hagit Sebban, Acco (IL)

Correspondence Address: KENYON & KENYON LLP, ONE BROADWAY, NEW YORK, NY, 10004, US

(21) Appl. No.: 11/788,771

(22) Filed: Apr. 19, 2007

Related U.S. Application Data
(60) Provisional application No. 60/793,495, filed on Apr. 19, 2006.
(60) Provisional application No. 60/802,121, filed on May 22, 2006.

(51) Int. Cl. A61K 31/54; A61K 31/535; A61P 7/00

(52) U.S. Cl. 514/223.5; 514/233.8

(57) ABSTRACT

Provided are stable pharmaceutical compositions comprising from about 2.5% to about 20% of a 2-aza-bicyclo[3.3.0]-octane-3-carboxylic acid derivative by weight of the composition and at least one pharmaceutically acceptable excipient, wherein the composition preferably has a total weight of less than 100 mg. Also provided are stable pharmaceutical compositions comprising a 2-aza-bicyclo[3.3.0]-octane-3-carboxylic acid derivative in a stabilizing-effective concentration and at least one pharmaceutically acceptable excipient. Further provided are methods for improving the stability of a pharmaceutical composition and methods for treating hypertension by administering a therapeutically effective amount of the stable pharmaceutical compositions of the invention.

### CROSS REFERENCE TO RELATED APPLICATIONS

This application claims the benefit of U.S. Ser. No. 60/793,495, filed Apr. 19, 2006, and Ser. No. 60/802,121, filed May 22, 2006, the contents of both of which are incorporated herein by reference in their entirety.

### FIELD OF THE INVENTION

This invention relates to stable compositions of 2-aza-bicyclo[3.3.0]-octane-3-carboxylic acid derivatives, and methods for their preparation.

### BACKGROUND OF THE INVENTION

Ramipril, quinapril, moexipril, enalapril, perindopril, and trandolapril are examples of 2-aza-bicyclo[3.3.0]-octane-3-carboxylic acid derivatives used in pharmaceutical formulations. Ramipril, which has the chemical name (2S,3aS,6aS)-1[(S)—N—[(S)-1-carboxy-3-phenyl-propyl]alanyl]octa hydrocyclopenta[b]pyrrole-2-carboxylic acid, 1-ethyl ester, is a pro-drug of ramiprilat, the active form of this angiotensin-converting enzyme (ACE) inhibitor.

Ramipril and certain other ACE inhibitors are effective antihypertensive drugs, but they are often susceptible to degradation. Ramipril degrades into two main products: diketopiperazine (DKP) and ramiprilat. Decomposition during manufacture and storage may adversely affect the effectiveness of the drug product or may cause the drug product to deviate from regulatory purity or potency requirements. It is therefore desirable to increase the stability of 2-aza-bicyclo[3.3.0]-octane-3-carboxylic acid derivative formulations.

The following figures illustrate the chemical structures of some examples of 2-aza-bicyclo[3.3.0]-octane-3-carboxylic acid derivatives and their corresponding active form degradants in addition to DKP.

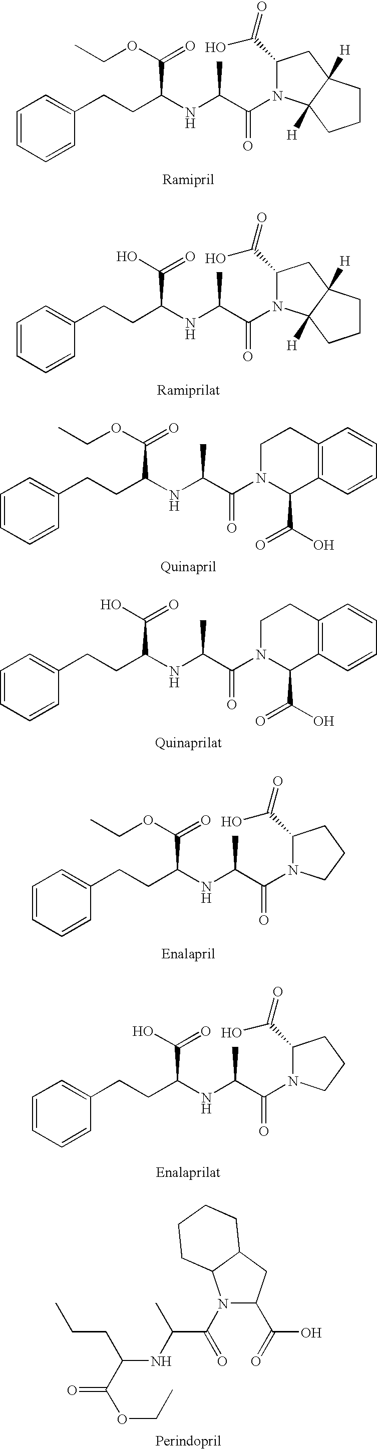

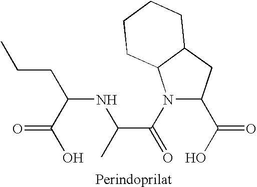

European Patent No. 280,999B1 and 317,878B1, U.S. Pat. Nos. 6,417,196; 4,830,853 and 4,793,998, and U.S. application Ser. No. 10/877,027, incorporated herein in their entirety, appear to address the stability of these derivatives, which highlights the continuing need to stabilize such compounds.

References in the field often teach the addition of various ingredients or process steps in order to improve the stability of the active ingredient. It is believed that the present invention can provide an additive or extra stabilizing effect, in addition to any stabilization that might be achieved by using additives or process steps taught in the art. For example, some commercially available tablets, such as King Pharmaceutical's Altace® tablets, are reportedly stabilized by an alkaline additive and/or cellulose-coated particles.

### SUMMARY OF THE INVENTION

The invention relates to stable pharmaceutical compositions comprising a 2-aza-bicyclo[3.3.0]-octane-3-carboxylic acid derivative and a minimal amount of inactive or non-therapeutic ingredients.

In one embodiment, the invention encompasses a stable pharmaceutical composition comprising from about 2.5% to about 20% of a 2-aza-bicyclo[3.3.0]-octane-3-carboxylic acid derivative by weight of the composition and at least one pharmaceutically acceptable excipient, wherein the composition preferably has a total weight of less than 100 mg.

In another embodiment, the invention encompasses a method for improving the stability of a pharmaceutical composition comprising combining a stabilizing-effective concentration of a 2-aza-bicyclo[3.3.0]-octane-3-carboxylic acid derivative with at least one pharmaceutically acceptable excipient, wherein the composition preferably has a total weight of less than 100 mg.

In another embodiment, the invention encompasses a stable pharmaceutical composition comprising a 2-aza-bicyclo[3.3.0]-octane-3-carboxylic acid derivative in a stabilizing-effective concentration and at least one pharmaceutically acceptable excipient.

The invention also encompasses a stable pharmaceutical composition comprising about 1.25 mg of ramipril and having a total weight of about 50 mg; comprising about 2.5 mg of ramipril and having a total weight of about 50 mg; comprising about 5 mg of ramipril and having a total weight of about 50 mg; or comprising about 10 mg of ramipril and having a total weight of about 50 mg.

In a preferred embodiment, the stable pharmaceutical compositions of the invention exhibit at least one, and preferably all, of the following characteristics:

(a) less than about 2% (preferably less than about 1%) of a diketopiperazine by weight of the derivative before degradation is present after storage at 40° C. under 75% relative humidity for one month;

(b) less than about 3% (preferably less than about 2%) of a diketopiperazine by weight of the derivative before degradation is present after storage at 40° C. under 75% relative humidity for two months;

(c) less than about 3.5% (preferably less than about 3%) of a diketopiperazine by weight of the derivative before degradation is present after storage at 40° C. under 75% relative humidity for three months;

(d) less than about 2% (preferably less than about 1%) by weight of the derivative is converted to diketopiperazine after storage at 40° C. under 75% relative humidity for one month;

(e) less than about 3% (preferably less than about 2%) by weight of the derivative is converted to diketopiperazine after storage at 40° C. under 75% relative humidity for two months; or

(f) less than about 3.5% (preferably less than about 3%) by weight of the derivative is converted to diketopiperazine after storage at 40° C. under 75% relative humidity for three months.

The invention further encompasses a method of treating hypertension in a mammal in need thereof comprising administering a therapeutically effective amount of the compositions of the invention.

### DETAILED DESCRIPTION OF THE INVENTION

The term “by weight,” unless otherwise specified, means by weight of the total composition.

The term “derivative” refers to a 2-aza-bicyclo[3.3.0]-octane-3-carboxylic acid derivative. 2-aza-bicyclo[3.3.0]-octane-3-carboxylic acid derivatives include, for example, ramipril, quinapril, moexipril, enalapril, perindopril, and trandolapril.

The term “DKP” refers to diketopiperazine. The term “active form degradant” refers to the active compounds that 2-aza-bicyclo[3.3.0]-octane-3-carboxylic acid derivatives degrade into. These derivatives tend to degrade into DKP and a corresponding active form degradant. For example, the active form degradant for ramipril is ramiprilat, quinaprilat for quinapril, moexipril for moexiprilat, enalaprilat for enalapril, perindoprilat for perindopril, trandolaprilat for trandolapril, and so forth.

The amount of the DKP present is determined as a percentage by weight of the derivative prior to degradation of the derivative. Stability of the compositions of the present invention can be characterized by either the total amount of DKP present after storage, or by the amount converted from the derivative to DKP after storage.

The invention relates to stable pharmaceutical compositions comprising a 2-aza-bicyclo[3.3.0]-octane-3-carboxylic acid derivative and at least one inactive or non-therapeutic ingredient. For example, for a composition comprising 1.25 mg of the derivative, greater stability is achieved at a 50 mg total weight (i.e., 2.5% of the derivative is present by weight of the total composition) than a composition having a 1.25 mg dose at a 100 mg total weight (1.25% of the derivative). Similarly, a composition comprising 2.5 mg of the derivative would exhibit greater stability at 50 mg total weight (5% of the derivative) than a composition having 2.5 mg dose at a 100 mg total weight (2.5% of the derivative), and so forth.

The stable pharmaceutical compositions of the invention resist degradation of the active ingredient when stored. For example, after exposure to “accelerated” storage conditions, such as at 40° C. and 70% relative humidity (RH), the invention demonstrates greater stability than pharmaceutical compositions that do not have from about 2.5% to about 20% of a 2-aza-bicyclo[3.3.0]-octane-3-carboxylic acid derivative by weight of the composition and at least one pharmaceutically acceptable excipient.

As used herein, a “stable” pharmaceutical composition is a pharmaceutical composition that exhibits at least one, and preferably all, of the following characteristics:

(a) less than about 2% of a diketopiperazine by weight of the derivative before degradation is present after storage at 40° C. under 75% relative humidity for one month;

(b) less than about 3% of a diketopiperazine by weight of the derivative before degradation is present after storage at 40° C. under 75% relative humidity for two months;

(c) less than about 3.5% of a diketopiperazine by weight of the derivative before degradation is present after storage at 40° C. under 75% relative humidity for three months;

(d) less than about 2% by weight of the derivative is converted to diketopiperazine after storage at 40° C. under 75% relative humidity for one month;

(e) less than about 3% by weight of the derivative is converted to diketopiperazine after storage at 40° C. under 75% relative humidity for two months; or

(f) less than about 3.5% by weight of the derivative is converted to diketopiperazine after storage at 40° C. under 75% relative humidity for three months.

The improved stability achieved by reducing the amount of inactive ingredients according to the present invention is apparent even in the absence of any specific ingredient intended to improve the stability of the active ingredient. Although not bound by any theory, it is believed that the stabilizing effect achieved by the present invention may be additive to any stabilizing effect that might be obtained by addition of any other stabilizing additives known in the art.

In one embodiment, the invention encompasses a stable pharmaceutical composition comprising from about 2.5% to about 20% of a 2-aza-bicyclo[3.3.0]-octane-3-carboxylic acid derivative by weight of the composition and at least one pharmaceutically acceptable excipient, wherein the composition preferably has a total weight of less than 100 mg.

Preferably, the stable pharmaceutical composition has a total weight of from about 50 mg to about 75 mg. More preferably, the composition has a total weight of about 50 mg.

Preferably, the stable pharmaceutical composition comprises about 2.5%, 5%, 10%, or 20% of a 2-aza-bicyclo[3.3.0]-octane-3-carboxylic acid derivative by weight of the composition.

The stable pharmaceutical composition may comprise about 1.25 mg, 2.5 mg, 5 mg, or 10 mg of the derivative. Preferably, the stable pharmaceutical composition comprising about 1.25 mg, 2.5 mg, 5 mg, or 10 mg of the derivative has a total weight of about 50 mg.

In a preferred embodiment, the stable pharmaceutical composition exhibits at least one, and preferably all, of the following characteristics:

(a) less than about 2% of a diketopiperazine by weight of the derivative before degradation is present after storage at 40° C. under 75% relative humidity for one month;

(b) less than about 3% of a diketopiperazine by weight of the derivative before degradation is present after storage at 40° C. under 75% relative humidity for two months; or

(c) less than about 3.5% of a diketopiperazine by weight of the derivative before degradation is present after storage at 40° C. under 75% relative humidity for three months.

Preferably, less than about 1%, more preferably less than about 0.5%, of a diketopiperazine by weight of the derivative before degradation is present after storage at 40° C. under 75% relative humidity for one month. Preferably, less than about 2%, more preferably less than about 1%, of a diketopiperazine by weight of the derivative before degradation is present after storage at 40° C. under 75% relative humidity for two months. Preferably, less than about 3%, more preferably less than about 2%, of a diketopiperazine by weight of the derivative before degradation is present after storage at 40° C. under 75% relative humidity for three months.

In a preferred embodiment, the stable pharmaceutical composition exhibits at least one, and preferably all, of the following characteristics:

(a) less than about 1% of a diketopiperazine by weight of the derivative before degradation is present after storage at 40° C. under 75% relative humidity for one month;

(b) less than about 2% of a diketopiperazine by weight of the derivative before degradation is present after storage at 40° C. under 75% relative humidity for two months; or

(c) less than about 3% of a diketopiperazine by weight of the derivative before degradation is present after storage at 40° C. under 75% relative humidity for three months.

In another preferred embodiment, the stable pharmaceutical composition exhibits at least one, and preferably all, of the following characteristics:

(a) less than about 2% by weight of the derivative is converted to diketopiperazine after storage at 40° C. under 75% relative humidity for one month;

(b) less than about 3% by weight of the derivative is converted to diketopiperazine after storage at 40° C. under 75% relative humidity for two months; or

(c) less than about 3.5% by weight of the derivative is converted to diketopiperazine after storage at 40° C. under 75% relative humidity for three months.

Preferably, less than about 1%, more preferably less than about 0.5% by weight of the derivative is converted to diketopiperazine after storage at 40° C. under 75% relative humidity for one month. Preferably, less than about 2%, more preferably less than about 1% by weight of the derivative is converted to diketopiperazine after storage at 40° C. under 75% relative humidity for two months. Preferably, less than about 3%, more preferably less than about 2% by weight of the derivative is converted to diketopiperazine after storage at 40° C. under 75% relative humidity for three months.

In a preferred embodiment, the stable pharmaceutical composition exhibits at least one, and preferably all, of the following characteristics:

(a) less than about 1% by weight of the derivative is converted to diketopiperazine after storage at 40° C. under 75% relative humidity for one month; or

(b) less than about 2% by weight of the derivative is converted to diketopiperazine after storage at 40° C. under 75% relative humidity for two months; or

(c) less than about 3% by weight of the derivative is converted to diketopiperazine after storage at 40° C. under 75% relative humidity for three months.

The 2-aza-bicyclo[3.3.0]-octane-3-carboxylic acid derivative may include at least one of ramipril, quinapril, moexipril, enalapril, perindopril, or trandolapril. Preferably, the derivative includes ramipril.

The stable pharmaceutical composition is generally in solid unit dosage form, for example in tablet, capsule, or powder form. Preferably, the stable pharmaceutical composition is in capsule form. When the stable pharmaceutical composition is in the form of a hard gelatin capsule, the weight of the hard gelatin capsule itself is not typically taken into account as part of the weight of the composition. Instead, the ingredients filled in to the capsule constitute the weight of the composition.

Preferably, the pharmaceutically acceptable excipient comprises at least one of pregelatinized starch, lactose anhydrous, povidone, or sodium stearyl fumarate. In a preferred embodiment, the stable pharmaceutical composition comprises pregelatinized starch and lactose anhydrous. Preferably, the pregelatinized starch and lactose anhydrous are present in about 1:5 to about 5:1 ratio. For example, the stable pharmaceutical composition can contain pregelatinized starch and lactose anhydrous each in about 20 to about 60 percent, preferably about 40 to about 60 percent, and more preferably about 50 percent by weight of the composition. Preferably, each of the pregelatinized starch and the lactose anhydrous are in the form of particles which pass through an about 150 micron screen. More preferably, about 90 to about 100 percent of the pregelatinized starch particles pass through about a 150 micron screen. More preferably, about 40 to about 65 percent of the lactose anhydrous particles pass through about a 150 micron screen. The presence of the pregelatinized starch particles and lactose anhydrous particles causes better flowability of the final blend and also increases the homogeneity of the active ingredient in the composition.

In one embodiment, the stable pharmaceutical composition comprises pregelatinized starch, lactose anhydrous, and sodium stearyl fumarate. In another embodiment, the stable pharmaceutical composition comprises pregelatinized starch, lactose anhydrous, povidone, and sodium stearyl fumarate.

The stable pharmaceutical composition may further comprise at least one additional active ingredient, e.g., a diuretic agent such as hydrochlorothiazide.

The invention also encompasses a stable pharmaceutical composition comprising a 2-aza-bicyclo[3.3.0]-octane-3-carboxylic acid derivative in a stabilizing-effective concentration and at least one pharmaceutically acceptable excipient, preferably wherein the composition has a total weight of less than 100 mg.

As used herein, a “stabilizing-effective concentration of a 2-aza-bicyclo[3.3.0]-octane-3-carboxylic acid derivative” is a concentration of the derivative that produces, in the absence of any stabilizing additives, a composition exhibiting at least one, and preferably all, of the following characteristics:

(a) less than about 2% of a diketopiperazine by weight of the derivative before degradation is present after storage at 40° C. under 75% relative humidity for one month;

(b) less than about 3% of a diketopiperazine by weight of the derivative before degradation is present after storage at 40° C. under 75% relative humidity for two months;

(c) less than about 3.5% of a diketopiperazine by weight of the derivative before degradation is present after storage at 40° C. under 75% relative humidity for three months;

(d) less than about 2% by weight of the derivative is converted to diketopiperazine after storage at 40° C. under 75% relative humidity for one month;

(e) less than about 3% by weight of the derivative is converted to diketopiperazine after storage at 40° C. under 75% relative humidity for two months; or

(f) less than about 3.5% by weight of the derivative is converted to diketopiperazine after storage at 40° C. under 75% relative humidity for three months.

The minimum stabilizing-effective concentration of a derivative depends on the total amount of the composition, with larger compositions generally requiring a higher percentage of derivative to achieve one or more of the degradation characteristics provided above. Whether a concentration of a derivative is a “stabilizing-effective concentration” can be easily determined by one of ordinary skill in the art by testing the degradation profile of the derivative in a composition free of any stabilizing additives, such as those exemplified herein.

In a preferred embodiment, less than about 1% of a diketopiperazine by weight of the derivative before degradation is present after storage at 40° C. under 75% relative humidity for one month. In another preferred embodiment, less than about 2% of a diketopiperazine by weight of the derivative before degradation is present after storage at 40° C. under 75% relative humidity for two months. In another preferred embodiment, less than about 3% of a diketopiperazine by weight of the derivative before degradation is present after storage at 40° C. under 75% relative humidity for three months.

In another preferred embodiment, less than about 1% by weight of the derivative is converted to diketopiperazine after storage at 40° C. under 75% relative humidity for one month. In another preferred embodiment, less than about 2% by weight of the derivative is converted to diketopiperazine after storage at 40° C. under 75% relative humidity for two months. In another preferred embodiment, less than about 3% by weight of the derivative is converted to diketopiperazine after storage at 40° C. under 75% relative humidity for three months.

Preferably, the stable pharmaceutical composition has a total weight of less than 100 mg, more preferably from about 50 mg to about 75 mg, and more preferably about 50 mg.

Preferably, the stable pharmaceutical composition comprises about 2.5%, 5%, 10%, or 20% of a 2-aza-bicyclo[3.3.0]-octane-3-carboxylic acid derivative by weight of the composition.

In a preferred embodiment, the stable pharmaceutical composition comprises about 1.25 mg, 2.5 mg, 5 mg, or 10 mg of the derivative. Preferably, the composition comprising about 1.25 mg, 2.5 mg, 5 mg, or 10 mg of the derivative has a total weight of about 50 mg.

The invention also encompasses methods for improving the stability of a pharmaceutical composition. In one embodiment, the invention encompasses a method for improving the stability of a pharmaceutical composition comprising combining a stabilizing-effective concentration of a 2-aza-bicyclo[3.3.0]-octane-3-carboxylic acid derivative with at least one pharmaceutically acceptable excipient, wherein the composition preferably has a total weight of less than 100 mg.

Preferably, the method for improving stability produces the stable pharmaceutical compositions of the invention. For example, the method may produce a composition where less than about 1% of a diketopiperazine by weight of the derivative before degradation is present after storage at 40° C. under 75% relative humidity for one month. Also preferably, the method produces a composition where less than about 2% of a diketopiperazine by weight of the derivative before degradation is present after storage at 40° C. under 75% relative humidity for two months. The method may also produce a composition where less than about 3% of a diketopiperazine by weight of the derivative before degradation is present after storage at 40° C. under 75% relative humidity for three months.

The method may also produce a composition where less than about 1% by weight of the derivative is converted to diketopiperazine after storage at 40° C. under 75% relative humidity for one month. Also preferably, the method produces a composition where less than about 2% by weight of the derivative is converted to diketopiperazine after storage at 40° C. under 75% relative humidity for two months. The method may also produce a composition where less than about 3% by weight of the derivative is converted to diketopiperazine after storage at 40° C. under 75% relative humidity for three months.

Preferably, the method produces a composition that exhibits at least one, and preferably all, of the following characteristics:

(a) less than about 2% of a diketopiperazine by weight of the derivative before degradation is present after storage at 40° C. under 75% relative humidity for one month;

(b) less than about 3% of a diketopiperazine by weight of the derivative before degradation is present after storage at 40° C. under 75% relative humidity for two months; or

(c) less than about 3.5% of a diketopiperazine by weight of the derivative before degradation is present after storage at 40° C. under 75% relative humidity for three months.

More preferably, the method produces a composition that exhibits at least one, and preferably all, of the following characteristics:

(a) less than about 1% of a diketopiperazine by weight of the derivative before degradation is present after storage at 40° C. under 75% relative humidity for one month;

(b) less than about 2% of a diketopiperazine by weight of the derivative before degradation is present after storage at 40° C. under 75% relative humidity for two months; or

(c) less than about 3% of a diketopiperazine by weight of the derivative before degradation is present after storage at 40° C. under 75% relative humidity for three months.

Preferably, the method produces a composition that exhibits at least one, and preferably all, of the following characteristics:

(a) less than about 2% by weight of the derivative is converted to diketopiperazine after storage at 40° C. under 75% relative humidity for one month;

(b) less than about 3% by weight of the derivative is converted to diketopiperazine after storage at 40° C. under 75% relative humidity for two months; or

(c) less than about 3.5% by weight of the derivative is converted to diketopiperazine after storage at 40° C. under 75% relative humidity for three months.

More preferably, the method produces a composition that exhibits at least one, and preferably all, of the following characteristics:

(a) less than about 1% by weight of the derivative is converted to diketopiperazine after storage at 40° C. under 75% relative humidity for one month;

(b) less than about 2% by weight of the derivative is converted to diketopiperazine after storage at 40° C. under 75% relative humidity for two months; or

(c) less than about 3% by weight of the derivative is converted to diketopiperazine after storage at 40° C. under 75% relative humidity for three months.

In another embodiment, the invention encompasses a stable pharmaceutical composition comprising about 1.25 mg of ramipril and having a total weight of about 50 mg; comprising about 2.5 mg of ramipril and having a total weight of about 50 mg; comprising about 5 mg of ramipril and having a total weight of about 50 mg; or comprising about 10 mg of ramipril and having a total weight of about 50 mg.

Any conventional method known in the art can be used to prepare the stable pharmaceutical compositions of the invention. Preferably, the method comprises at least one of dry mixing, dry granulation or wet granulation.

The composition is typically processed into solid dosage form, preferably in the form of a tablet, capsule, or powder. Conventional tableting processes can be employed, e.g., by forming a tablet from a desired mixture of ingredients into the appropriate shape using a conventional tablet press. Tablet formulation and processing techniques are generally known in the field. Capsule formulation methods are also commonly known in the art.

The stable pharmaceutical compositions of the present invention can also contain inactive ingredients such as diluents, carriers, fillers, bulking agents, binders, disintegrants, disintegration inhibitors, absorption accelerators, wetting agents, lubricants, glidants, surface active agents, flavoring agents, preservatives, antioxidants, buffering agents, and any other excipient commonly used in the pharmaceutical industry.

Diluents increase the bulk of a solid pharmaceutical composition, and may make a pharmaceutical dosage form containing the composition easier for the patient and care giver to handle. Diluents used in the composition include diluents commonly used in solid pharmaceutical compositions. Diluents include, but are not limited to, calcium carbonate, calcium phosphate (dibasic or tribasic), calcium sulfate, dextrates, dextrin, dextrose excipient, fructose, kaolin, lactitol, anhydrous lactose, lactose monohydrate, maltose, mannitol, sorbitol, sucrose, starch, pregelatinized starch, talc and the like.

Carriers for use in the compositions may include, but are not limited to, lactose, white sugar, sodium chloride, glucose, urea, additional starch, calcium carbonate, kaolin, crystalline cellulose, silicic acid, and the like.

Binders help to bind the active ingredient and other excipients together. Binders used in the composition include binders commonly used in solid pharmaceutical compositions. Binders include, but are not limited to, acacia, alginic acid, carbomer, sodium carboxymethylcellulose, dextrin, ethylcellulose, gelatin, glucose, guar gum, hydroxypropylcellulose, maltose, methylcellulose, povidone, starch, gelatin, methylcellulose, polyethylene oxide and the like.

Disintegrants can increase dissolution. Examples of suitable disintegrants are starch, pregelatinized starch, sodium starch glycolate, sodium carboxymethylcellulose, crosslinked sodium carboxymethylcellulose (e.g., sodium croscarmellose; crosslinked starch available under the registered trademark Ac-Di-Sol from FMC Corp., Philadelphia, Pa.), clays (e.g., magnesium aluminum silicate), microcrystalline cellulose (such as those available under the registered trademark Avicel from FMC Corp. or the registered trademark Emcocel from Mendell Corp., Carmel, N.Y.), alginates, gums, surfactants, effervescent mixtures, hydrous aluminum silicate, cross-linked polyvinylpyrrolidone (available commercially under the registered trademark PVP-XL from International Specialty Products, Inc.), and others as known in the art.

Disintegration inhibitors may include, but are not limited to, white sugar, stearin, coconut butter, hydrogenated oils, and the like. Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

A lubricant can be added to the composition for ease in processing, e.g., to reduce adhesion to the equipment used during processing, and to ease release of the product from a punch or dye during tableting. Lubricants used in the composition include those commonly used in solid pharmaceutical compositions, including, e.g., calcium stearate, glyceryl behenate, magnesium stearate, mineral oil, polyethylene glycol, sodium stearyl fumarate, stearic acid, talc, vegetable oil, sodium lauryl sulfate, and zinc stearate.

Glidants can be added to improve the flowability of a non-compacted solid composition and improve the accuracy of dosing. Glidants used in the composition include glidants commonly used in solid pharmaceutical compositions, including, e.g., colloidal silicon dioxide, magnesium trisilicate, powdered cellulose, starch, talc, and tribasic calcium phosphate.

Flavoring agents and flavor enhancers make the dosage form more palatable to the patient. Common flavoring agents and flavor enhancers for pharmaceutical products that can be included in the composition of the present invention include for example maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

The compositions can also be dyed using any pharmaceutically acceptable colorant to improve their appearance and/or facilitate patient identification of the product and unit dosage level. Coloring agents may include titanium dioxide and/or dyes suitable for food such as those known as FD & C dyes and natural coloring agents such as grape skin extract, beet red powder, beta carotene, annato, carmine, turmeric, paprika, and so forth.

Sweetening agents such as sorbitol, saccharin, sodium saccharin, sucrose, aspartame, fructose, mannitol and invert sugar can be added to improve the taste. Preservatives and chelating agents such as alcohol, sodium benzoate, butylated hydroxy toluene, butylated hydroxyanisole and ethylenediamine tetraacetic acid can be added at safe levels to improve storage stability.

As described above, the compositions of the invention can be prepared by dry mixing, dry granulation or wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tableted or other excipients can be added prior to tableting, such as a glidant and/or a lubricant. Preferred dosage forms of the invention include a tablet, capsule, or powder.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule can be prepared conventionally such as by blending. A capsule filling of the present invention can comprise any of the aforementioned blends and granulates that are described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used include, but are not limited to, gum arabic powder, tragacanth gum powder, gelatin, ethanol, and the like. Disintegrating agents used include, but are not limited to, agar, laminalia, and the like.

The invention also encompasses a method of treating hypertension in a mammal in need thereof comprising administering a therapeutically effective amount of the compositions of the invention. The amount of the derivative or pharmaceutically acceptable salt thereof contained in a composition of the invention for treating hypertension is not specifically restricted; however, the dose should be sufficient to treat, ameliorate, or reduce the condition. The dosage of a pharmaceutical composition for treating hypertension according to the present invention will depend on the method of use, the age, sex, weight and condition of the patient.

Having described the invention with reference to certain preferred embodiments, other embodiments will become apparent to one skilled in the art from consideration of the specification. The invention is further defined by reference to the following examples describing in detail the analysis of the crystals and processes for making the crystals of the invention. It will be apparent to those skilled in the art that many modifications, both to materials and methods, may be practiced without departing from the scope of the invention.

### EXAMPLES

### Examples 1-2

### Ramipril 1.25 mg Capsules

The ingredients of Table 1 are blended and filled into hard gelatin capsules. The capsule shell contains gelatin, titanium dioxide, and colorant.

TABLE 1

| | | Example 2 |
|---|---|---|
| | Example 1 | (comparative) |
| Ingredient | mg/capsule | mg/capsule |
| Ramipril | 1.25 | 1.25 |
| Pregelatinized Starch | 23.35 | 47.95 |
| Lactose Anhydrous | 25.0 | 50.0 |
| Sodium Stearyl Fumarate | 0.4 | 0.8 |
| Total | 50.0 | 100.0 |

### Examples 3-4

### Ramipril 2.5 mg Capsules

The ingredients listed in Table 2 are blended and filled into capsules.

TABLE 2

| | | Example 4 |
|---|---|---|
| | Example 3 | (comparative) |
| Ingredient | mg/tablet | mg/tablet |
| Ramipril | 2.5 | 2.5 |
| Pregelatinized Starch | 22.1 | 46.7 |
| Lactose Anhydrous | 25.0 | 50.0 |
| Sodium Stearyl Fumarate | 0.4 | 0.8 |
| Total | 50.0 | 100.0 |

### Examples 5-6

### Trandolapril 1 mg Tablets

The ingredients listed in Table 3 are blended and compressed into tablets.

TABLE 3

| | | Example 6 |
|---|---|---|
| | Example 5 | (comparative) |
| Ingredient | mg/tablet | mg/tablet |
| Trandolapril | 1.0 | 1.0 |
| Pregelatinized Starch | 10 | 20.0 |
| Lactose Anhydrous | 35.7 | 72.4 |
| Povidone | 2.5 | 5.0 |
| Color | 0.05 | 0.1 |
| Sodium Stearyl Fumarate | 0.75 | 1.5 |
| Total | 50.0 | 100.0 |

Stability Results

A stability test was performed by packing the capsules of Example 1, Example 2, and commercially available Altace® 1.25 mg and 2.5 mg capsules in HDPE (high-density polyethylene) bottles and storing them at 40° C. under 75% relative humidity.

According to the Physician's Desk Reference 2006 ed., the inactive ingredients in Altace are pregelatinized starch NF, gelatin, and titanium dioxide. Altace 1.25 mg is supplied in a yellow, hard gelatin capsule and the shell contains yellow iron oxide. Altace 2.5 is supplied in an orange, hard gelatin capsule and the shell contains D&C yellow # 10 and FD&C red # 40. The total weight of Altace 1.25 mg and 2.5 mg capsules were measured to be 125 mg.

| | | | Altace | Altace |
|---|---|---|---|---|
| | Example 1 | Example 2 | 1.25 mg | 2.5 mg |
| Ramipril (mg) | 1.25 | 1.25 | 1.25 | 2.5 |
| Total Weight (mg) | 50.0 | 100.0 | 125 | 125 |

After storage for 1, 2 and 3 months (M), the amount of ramiprilat and DKP present was measured by high performance liquid chromatography (HPLC) using the following parameters:

Column: Zorbax SB C-8, 5 μm, 250×4.6 mm

Mobile Phase Buffer adjusted to pH 2.00 with acetonitrile (65:35 V/V)

Flow Rate: 1.0 mL/min

Detection: UV, λ=215 nm

Column Temp.: 60° C.

Sample Temp.: 4° C.

Injection Volume: 50 μl

Stability results are shown in Tables 4 and 5.

TABLE 4
Ramipril Capsule Formulations - Total Amount of DKP

| | Total amount | Total amount | Total amount |
|---|---|---|---|
| | of DKP | of DKP | of DKP |
| Example | after 1M (%) | after 2M (%) | after 3M (%) |
| 1 | 1.0 | 2.0 | 3.5 |
| 1.25 mg | | | |
| 2 | 2.9 | 5.9 | 8.3 |
| 1.25 mg | | | |
| Altace | 2.5 | 3.7 | 4.9 |
| 1.25 mg | | | |
| Altace | 2.0 | 2.8 | 3.7 |
| 2.5 mg | | | |

The amount of DKP converted is extrapolated by subtracting the estimated initial DKP amount from the total amount of DKP present after storage.

TABLE 5
Ramipril Capsule Formulations - Amount of DKP Converted

| | | Amount | Amount | Amount |
|---|---|---|---|---|
| | | converted | converted | converted |
| | Initial DKP | after | after | after |
| Example | Amount (%) | 1M (%) | 2M (%) | 3M (%) |
| 1 | 0.07 | 0.9 | 1.9 | 3.4 |
| 1.25 mg | | | | |
| 2 | 0.07 | 2.8 | 5.8 | 8.2 |
| 1.25 mg | | | | |
| Altace | 1.2 | 1.3 | 2.5 | 3.7 |
| 1.25 mg | | | | |
| Altace | 1.0 | 1.0 | 1.8 | 2.7 |
| 2.5 mg | | | | |

As shown in Tables 4 and 5, Example 1 having 2.5% by weight of ramipril contains less DKP than Example 2, which contains 1.25% by weight of ramipril. In addition, the amount of DKP converted after storage is much less in Example 1 than in Example 2. Therefore, the amount of DKP present and the amount of DKP converted are reduced when the active ingredient in Example 1 constitutes a larger proportion of the composition than Example 2, even when the ingredients are identical.

1. A stable pharmaceutical composition comprising from about 2.5% to about 20% of a 2-aza-bicyclo[3.3.0]-octane-3-carboxylic acid derivative by weight of the composition and at least one pharmaceutically acceptable excipient, wherein the composition has a total weight of less than 100 mg.

2. The stable pharmaceutical composition of claim 1, wherein the composition has a total weight of from about 50 mg to about 75 mg.

3. The stable pharmaceutical composition of claim 1, wherein the composition has a total weight of about 50 mg.

4. The stable pharmaceutical composition of claim 1 comprising about 2.5% of a 2-aza-bicyclo[3.3.0]-octane-3-carboxylic acid derivative by weight of the composition.

5. The stable pharmaceutical composition of claim 1 comprising about 5% of a 2-aza-bicyclo[3.3.0]-octane-3-carboxylic acid derivative by weight of the composition.

6. The stable pharmaceutical composition of claim 1 comprising about 10% of a 2-aza-bicyclo[3.3.0]-octane-3-carboxylic acid derivative by weight of the composition.

7. The stable pharmaceutical composition of claim 1, wherein the composition comprises about 1.25 mg, 2.5 mg, 5 mg, or 10 mg of the derivative.

8. The stable pharmaceutical composition of claim 1, wherein the composition exhibits at least one of the following characteristics:
(a) less than about 2% of a diketopiperazine by weight of the derivative before degradation is present after storage at 40° C. under 75% relative humidity for one month;
(b) less than about 3% of a diketopiperazine by weight of the derivative before degradation is present after storage at 40° C. under 75% relative humidity for two months; or
(c) less than about 3.5% of a diketopiperazine by weight of the derivative before degradation is present after storage at 40° C. under 75% relative humidity for three months.

9. The stable pharmaceutical composition of claim 1, wherein the composition exhibits at least one of the following characteristics:
(a) less than about 1% of a diketopiperazine by weight of the derivative before degradation is present after storage at 40° C. under 75% relative humidity for one month;
(b) less than about 2% of a diketopiperazine by weight of the derivative before degradation is present after storage at 40° C. under 75% relative humidity for two months; or
(c) less than about 3% of a diketopiperazine by weight of the derivative before degradation is present after storage at 40° C. under 75% relative humidity for three months.

10. The stable pharmaceutical composition of claim 1, wherein:
(a) less than about 2% of a diketopiperazine by weight of the derivative before degradation is present after storage at 40° C. under 75% relative humidity for one month;
(b) less than about 3% of a diketopiperazine by weight of the derivative before degradation is present after storage at 40° C. under 75% relative humidity for two months; and
(c) less than about 3.5% of a diketopiperazine by weight of the derivative before degradation is present after storage at 40° C. under 75% relative humidity for three months.

11. The stable pharmaceutical composition of claim 1, wherein the composition exhibits at least one of the following characteristics:
(a) less than about 2% by weight of the derivative is converted to diketopiperazine after storage at 40° C. under 75% relative humidity for one month;
(b) less than about 3% by weight of the derivative is converted to diketopiperazine after storage at 40° C. under 75% relative humidity for two months; or
(c) less than about 3.5% by weight of the derivative is converted to diketopiperazine after storage at 40° C. under 75% relative humidity for three months.

12. The stable pharmaceutical composition of claim 1, wherein the composition exhibits at least one of the following characteristics:
(a) less than about 1% by weight of the derivative is converted to diketopiperazine after storage at 40° C. under 75% relative humidity for one month; or
(b) less than about 2% by weight of the derivative is converted to diketopiperazine after storage at 40° C. under 75% relative humidity for two months; or
(c) less than about 3% by weight of the derivative is converted to diketopiperazine after storage at 40° C. under 75% relative humidity for three months.

13. The stable pharmaceutical composition of claim 1, wherein:
(a) less than about 2% by weight of the derivative is converted to diketopiperazine after storage at 40° C. under 75% relative humidity for one month;
(b) less than about 3% by weight of the derivative is converted to diketopiperazine after storage at 40° C. under 75% relative humidity for two months; and
(c) less than about 3.5% by weight of the derivative is converted to diketopiperazine after storage at 40° C. under 75% relative humidity for three months.

14. The stable pharmaceutical composition of claim 1, wherein the 2-aza-bicyclo[3.3.0]-octane-3-carboxylic acid derivative includes at least one of ramipril, quinapril, moexipril, enalapril, perindopril, or trandolapril.

15. The stable pharmaceutical composition of claim 1, wherein the 2-aza-bicyclo[3.3.0]-octane-3-carboxylic acid derivative includes ramipril.

16. The stable pharmaceutical composition of claim 1, wherein the composition is in tablet, capsule, or powder form.

17. The stable pharmaceutical composition of claim 1, wherein the composition is in capsule form.

18. The stable pharmaceutical composition of claim 1, wherein the pharmaceutically acceptable excipient is at least one of pregelatinized starch, lactose anhydrous, povidone, or sodium stearyl fumarate.

19. The stable pharmaceutical composition of claim 1, wherein the composition comprises pregelatinized starch, lactose anhydrous, and sodium stearyl fumarate.

20. The stable pharmaceutical composition of claim 1, wherein the composition further comprises at least one diuretic agent.

21. The stable pharmaceutical composition of claim 1, wherein the composition further comprises hydrochlorothiazide.

22. The stable pharmaceutical composition of claim 1, wherein:
(a) less than about 1% of a diketopiperazine by weight of the derivative before degradation is present after storage at 40° C. under 75% relative humidity for one month;
(b) less than about 2% of a diketopiperazine by weight of the derivative before degradation is present after storage at 40° C. under 75% relative humidity for two months; and
(c) less than about 3% of a diketopiperazine by weight of the derivative before degradation is present after storage at 40° C. under 75% relative humidity for three months.

23. The stable pharmaceutical composition of claim 1, wherein:
(a) less than about 1% by weight of the derivative is converted to diketopiperazine after storage at 40° C. under 75% relative humidity for one month;
(b) less than about 2% by weight of the derivative is converted to diketopiperazine after storage at 40° C. under 75% relative humidity for two months; and
(c) less than about 3% by weight of the derivative is converted to diketopiperazine after storage at 40° C. under 75% relative humidity for three months.

24. A method for improving the stability of a pharmaceutical composition comprising combining a stabilizing-effective concentration of a 2-aza-bicyclo[3.3.0]-octane-3-carboxylic acid derivative with at least one pharmaceutically acceptable excipient, wherein the composition has a total weight of less than 100 mg.

25. The method of claim 24, wherein the composition comprises about 2.5% of a 2-aza-bicyclo[3.3.0]-octane-3-carboxylic acid derivative by weight of the composition.

26. The method of claim 24, wherein the composition comprises about 1.25 mg, 2.5 mg, 5 mg, or 10 mg of the derivative.

27. The method of claim 24, wherein the composition exhibits at least one of the following characteristics:
(a) less than about 2% of a diketopiperazine by weight of the derivative before degradation is present after storage at 40° C. under 75% relative humidity for one month;
(b) less than about 3% of a diketopiperazine by weight of the derivative before degradation is present after storage at 40° C. under 75% relative humidity for two months; or
(c) less than about 3.5% of a diketopiperazine by weight of the derivative before degradation is present after storage at 40° C. under 75% relative humidity for three months.

28. The method of claim 24, wherein the composition exhibits at least one of the following characteristics:
(a) less than about 1% of a diketopiperazine by weight of the derivative before degradation is present after storage at 40° C. under 75% relative humidity for one month;
(b) less than about 2% of a diketopiperazine by weight of the derivative before degradation is present after storage at 40° C. under 75% relative humidity for two months; or
(c) less than about 3% of a diketopiperazine by weight of the derivative before degradation is present after storage at 40° C. under 75% relative humidity for three months.

29. The method of claim 24, wherein:
(a) less than about 2% of a diketopiperazine by weight of the derivative before degradation is present after storage at 40° C. under 75% relative humidity for one month;
(b) less than about 3% of a diketopiperazine by weight of the derivative before degradation is present after storage at 40° C. under 75% relative humidity for two months; and
(c) less than about 3.5% of a diketopiperazine by weight of the derivative before degradation is present after storage at 40° C. under 75% relative humidity for three months.

30. The method of claim 24, wherein:
(a) less than about 1% of a diketopiperazine by weight of the derivative before degradation is present after storage at 40° C. under 75% relative humidity for one month;
(b) less than about 2% of a diketopiperazine by weight of the derivative before degradation is present after storage at 40° C. under 75% relative humidity for two months; and
(c) less than 3% of a diketopiperazine by weight of the derivative before degradation is present after storage at 40° C. under 75% relative humidity for three months.

31. The method of claim 24, wherein the composition exhibits at least one of the following characteristics:
(a) less than about 2% by weight of the derivative is converted to diketopiperazine after storage at 40° C. under 75% relative humidity for one month;
(b) less than about 3% by weight of the derivative is converted to diketopiperazine after storage at 40° C. under 75% relative humidity for two months; or
(c) less than about 3.5% by weight of the derivative is converted to diketopiperazine after storage at 40° C. under 75% relative humidity for three months.

32. The method of claim 24, wherein the composition exhibits at least one of the following characteristics:
(a) less than about 1% by weight of the derivative is converted to diketopiperazine after storage at 40° C. under 75% relative humidity for one month;
(b) less than about 2% by weight of the derivative is converted to diketopiperazine after storage at 40° C. under 75% relative humidity for two months; or
(c) less than about 3% by weight of the derivative is converted to diketopiperazine after storage at 40° C. under 75% relative humidity for three months.

33. The method of claim 24, wherein:
(a) less than about 2% by weight of the derivative is converted to diketopiperazine after storage at 40° C. under 75% relative humidity for one month;
(b) less than about 3% by weight of the derivative is converted to diketopiperazine after storage at 40° C. under 75% relative humidity for two months; and
(c) less than about 3.5% by weight of the derivative is converted to diketopiperazine after storage at 40° C. under 75% relative humidity for three months.

34. The method of claim 24, wherein:
(a) less than about 1% by weight of the derivative is converted to diketopiperazine after storage at 40° C. under 75% relative humidity for one month;
(b) less than about 2% by weight of the derivative is converted to diketopiperazine after storage at 40° C. under 75% relative humidity for two months; and
(c) less than about 3% by weight of the derivative is converted to diketopiperazine after storage at 40° C. under 75% relative humidity for three months.

35. The method of claim 24, wherein the method comprises at least one of dry mixing, dry granulation or wet granulation.

36. A stable pharmaceutical composition prepared by the method of claim 24.

37. A stable pharmaceutical composition comprising a 2-aza-bicyclo[3.3.0]-octane-3-carboxylic acid derivative in a stabilizing-effective concentration and at least one pharmaceutically acceptable excipient, wherein:
(a) less than about 2% of a diketopiperazine by weight of the derivative before degradation is present after storage at 40° C. under 75% relative humidity for one month;
(b) less than about 3% of a diketopiperazine by weight of the derivative before degradation is present after storage at 40° C. under 75% relative humidity for two months; and
(c) less than about 3.5% of a diketopiperazine by weight of the derivative before degradation is present after storage at 40° C. under 75% relative humidity for three months.

38. The stable pharmaceutical composition of claim 37, wherein:
(a) less than about 2% by weight of the derivative is converted to diketopiperazine after storage at 40° C. under 75% relative humidity for one month;
(b) less than about 3% by weight of the derivative is converted to diketopiperazine after storage at 40° C. under 75% relative humidity for two months; and
(c) less than about 3.5% by weight of the derivative is converted to diketopiperazine after storage at 40° C. under 75% relative humidity for three months.

39. The stable pharmaceutical composition of claim 37, wherein:
(a) less than about 1% of a diketopiperazine by weight of the derivative before degradation is present after storage at 40° C. under 75% relative humidity for one month;
(b) less than about 2% of a diketopiperazine by weight of the derivative before degradation is present after storage at 40° C. under 75% relative humidity for two months; and
(c) less than about 3% of a diketopiperazine by weight of the derivative before degradation is present after storage at 40° C. under 75% relative humidity for three months.

40. The stable pharmaceutical composition of claim 37, wherein:
(a) less than about 1% by weight of the derivative is converted to diketopiperazine after storage at 40° C. under 75% relative humidity for one month;
(b) less than about 2% by weight of the derivative is converted to diketopiperazine after storage at 40° C. under 75% relative humidity for two months; and
(c) less than about 3% by weight of the derivative is converted to diketopiperazine after storage at 40° C. under 75% relative humidity for three months.

41. A stable pharmaceutical composition comprising about 1.25 mg of ramipril, wherein the composition has a total weight of about 50 mg.

42. A stable pharmaceutical composition comprising about 2.5 mg of ramipril, wherein the composition has a total weight of about 50 mg.

43. A stable pharmaceutical composition comprising about 5 mg of ramipril, wherein the composition has a total weight of about 50 mg.

44. A stable pharmaceutical composition comprising about 10 mg of ramipril, wherein the composition has a total weight of about 50 mg.

45. A method of treating hypertension in a mammal in need thereof comprising administering a therapeutically effective amount of the composition of claim 1.
